(19) United States
(12) Patent Application Publication
(10) Pub. No.: US 2020/0264191 A1
(43) Pub. Date: Aug. 20, 2020

(54) METHODS AND KITS FOR DIAGNOSIS OF FAMILIAL MEDITERRANEAN FEVER

(71) Applicants: INSERM (Institut National de la Santé et de la Recherche Médicale), Paris (FR); Université Claude Bernard - Lyon 1, Villeurbanne (FR); Ecole Normale Supérieure de Lyon, Lyon (FR); Centre National de la Recherche Scientifique (CNRS), Paris (FR); Hospices Civils de Lyon, Lyon (FR)

(72) Inventors: Thomas HENRY, Lyon Cedex 07 (FR); Yvan JAMILLOUX, Lyon Cedex 07 (FR); Lucie LEFEUVRE, Lyon (FR); Amandine MARTIN, Lyon Cedex 07 (FR); Flora MAGNOTTI, Lyon Cedex 07 (FR); Alexandre BELOT, Lyon Cedex 07 (FR)

(21) Appl. No.: 16/645,191

(22) PCT Filed: Sep. 6, 2018
(86) PCT No.: PCT/EP2018/074053

§ 371 Date: Mar. 6, 2020

(30) Foreign Application Priority Data
Sep. 7, 2017 (EP) 17306158.1

(51) Int. Cl. G01N 33/68; G01N 33/564; G01N 33/50

(52) U.S. Cl. CPC G01N 33/6869; G01N 33/564; G01N 2333/545; G01N 2800/7095; G01N 2800/24; G01N 33/5047

(57) ABSTRACT

The present invention relates to a non-invasive, specific and rapid diagnostic method of Familial Mediterranean fever (FMF) in a subject said method comprising the step of measuring the level of cytokine (IL-18 or IL-1 beta) secreted by immune primary cells (or cell death level of these cells) obtained from said subject, which have been beforehand treated with a Protein Kinase C (PKC) inhibitor, and optionally beforehand treated with a NF-kB activator such as LPS. Inventors show based on the extensive study of the inflammasome process of the monocytes, that PKC superfamily inhibitors trigger inflammasome activation in monocytes from FMF patients while they are not sufficient to do so in monocytes from healthy donors (HD) or from patient having hyperimmunoglobulinemia D syndrome (HID S). Using cytokine release quantification or determination of real time cell death kinetics, inventors demonstrate that PKC superfamily inhibitors can discriminate FMF patients from HD or from patients with systemic inflammation from other aetiologies. These results thus set-up the basis for the development of a rapid functional specific diagnostic test for FMF. Methods of treatment are disclosed.

### FIELD OF THE INVENTION

The present invention relates to methods and kits for diagnosis of Familial Mediterranean Fever. More specifically present invention relates to methods and kits for diagnosis of Familial Mediterranean Fever using protein Kinase C superfamily inhibitors.

### BACKGROUND OF THE INVENTION

Familial Mediterranean Fever (FMF) is the most frequent hereditary systemic autoinflammatory disorder characterized by short and recurrent episodes of fever and chest/abdominal pain (Sonmez, Batu, and Ozen 2016). Its main complication is secondary amyloidosis, which can lead to renal failure. Its prevalence is highly variable worldwide varying from 1:150 to 1:10,000. In several countries including Turkey, Italy, Israel, Armenia and Japan, FMF is not considered a rare disease (based on the European definition of a prevalence<1:2,000) (Migita et al. 2016; La Regina et al. 2003; Ozen et al. 1998; Ben-Chetrit and Touitou 2009; Daniels et al. 1995; Sarkisian et al. 2008). Colchicine is the main treatment for FMF and is highly efficient in most patients to prevent acute inflammation and amyloidosis by decreasing chronic subclinical inflammation (Goldfinger 1972). Daily and lifelong administration of colchicine is currently recommended for FMF patients.

FMF diagnosis relies first on clinical criteria (e.g. Livneh, Tel hashomer and Yalcinkaya-Ozen criteria) (Giancane et al. 2015; Demirkaya et al. 2016). Due to the absence of pathognomonic clinical symptoms, to heterogeneity in clinical presentations (Padeh et al. 2010; Mor, Gal, and Livneh 2003), FMF diagnosis can be challenging (Giancane et al. 2015). As of today, genetic screening is performed to get a definitive confirmation of the diagnosis. FMF is associated with mutations in the MEFV gene. Mendelian transmission of the disease occurs in an autosomal recessive mode. Nine sequence variants of MEFV are considered clearly pathogenic (Shinar et al. 2012). Confirmation from genetic testing is unambiguous in the case of patients homozygous or compound heterozygous for two clearly pathogenic MEFV variants. Yet, there are 317 MEFV variants listed in the Infevers database (Sarrauste de Menthiere et al. 2003) most of them of uncertain clinical significance, which can result in misdiagnosis or diagnosis delay (Lidar et al. 2005). Furthermore, a substantial proportion of clinically diagnosed FMF patients (10 to 30% depending on the studies) present only a single MEFV pathogenic variant (Dode et al. 2000; Jeru et al. 2013; Lachmann et al. 2006). Finally, no MEFV pathogenic variant is found in 5 to 14% of clinically-diagnosed FMF patients (Lachmann et al. 2006; Toplak et al. 2012). Further increasing the complexity of the genotype-phenotype correlations, some individuals in whom two clearly pathogenic variants are identified do not present neither symptoms of FMF nor symptoms of amyloidosis (referred to as phenotype III) (Camus et al. 2012) (Lachmann et al. 2006) (Kogan et al. 2001). Due to all these situations, genetic testing has only a 70-80% positive predictive value (Soriano and Manna 2012) and the median delay between disease onset and diagnosis is still relatively long (1.4 years for patients born in the 21st century)(Toplak et al. 2012).

MEFV encodes Pyrin, an inflammasome sensor detecting Rho GTPases activity modifications. Inactivation of RhoGTPases by various bacterial toxins triggers assembly of the Pyrin inflammasome resulting in caspase-1 activation, secretion of the pro-inflammatory cytokine IL-1β and triggering of an inflammatory cell death termed pyroptosis (Martinon, Burns, and Tschopp 2002; Cookson and Brennan 2001; Xu et al. 2014). At steady state, Pyrin is phosphorylated by two kinases (PKN1/2) from the PKC superfamily. Phosphorylated Pyrin is sequestered through interaction with 14-3-3 chaperone proteins (Jeru et al. 2005; Masters et al. 2016; Park et al. 2016) (Gao et al. 2016). Rho GTPases modification leads to dephosphorylation of Pyrin. Dephosphorylation of Pyrin provokes its release from the 14-3-3 protein and the assembly/activation of the Pyrin inflammasome. Of note, in healthy individuals, the switch from the free Pyrin to a mature Pyrin inflammasome is under the control of the microtubule dynamics (Gao et al. 2016). Colchine, a microtubule destabilizing drug, specifically blocks the Pyrin inflammasome at this late step post-Pyrin release from the 14-3-3 proteins (Gao et al. 2016). In FMF patients, this second regulatory mechanism might be deficient since a recent report indicated that colchicine is inefficient to block Pyrin inflammasome activation in PBMCs from FMF patients (Van Gorp et al. 2016). A two steps-activation based model is thus emerging with i) a dephosphorylation of Pyrin likely associated with inhibition of PKN1/2, and ii) a Pyrin inflammasome maturation step involving a colchicine-targetable microtubule dynamics event (Gao et al. 2016).

WO2017/042381 discloses a diagnostic method to identify a subject suffering from FMF, without treating immune primary cells obtained from the subject with a Protein Kinase C (PKC) inhibitor. Jamilloux et al. 2016 discloses the activation, the signaling, the regulation of interleukin-1 and describes the autoinflammatory diseases or related-diseases where the pathological role of interleukin-1 has been demonstrated.

Accordingly, there remains an unmet need in the art for specific and more rapid diagnostic test for FMF, reflecting directly the activation of Pyrin inflammasome process of monocytes.

The inventors therefore set up a diagnostic method of FMF that allows to directly reflect the activation of Pyrin inflammasome process in monocytes.

In this invention, inventors demonstrate that PKC (Protein Kinase C) superfamily inhibitors specifically trigger differential inflammasome activation in monocytes from FMF patients but not in other systemic inflammation from other aetiologies such as hyperimmunoglobulinemia D syndrome (HIDS).

### SUMMARY OF THE INVENTION

Here inventors identified, based on the extensive study of the Pyrin inflammasome process of the monocytes, that PKC superfamily inhibitors trigger Pyrin inflammasome activation in monocytes from FMF patients while they are not sufficient to do so in monocytes from healthy donors (HD) or from HIDS patient. Using IL-1β release quantification or determination of real time cell death kinetics, inventors demonstrate that PKC superfamily inhibitors can discriminate FMF patients from HD or from patients with systemic inflammation from other aetiologies. Similarly, IL-18, which is released in an inflammasome-dependent manner as IL-1β, can be used to discriminate FMF patients from HD or from patients systemic inflammation from other aetiologies. These results thus set-up the basis for the development of a rapid functional specific diagnostic test for FMF.

Thus, the present invention relates to an in vitro method for diagnosing Familial Mediterranean Fever (FMF) disease in a subject, comprising the steps of i) treating immune primary cells obtained from the subject and beforehand treated with a NF-κB activator, with a Protein Kinase C (PKC) inhibitor, ii) detecting the level of IL-1β secreted from these cells supernatant iii) comparing the level determined in step ii) with a reference value and iv) concluding that the subject suffers from an Familial Mediterranean Fever when the IL1 beta level determined at step ii) is higher than the reference value.

The present invention relates to an in vitro method for diagnosing Familial Mediterranean Fever (FMF) disease in a subject, comprising the steps of i) treating immune primary cells obtained from the subject, with a Protein Kinase C (PKC) inhibitor ii) detecting the level of IL-18 from these cells supernatant iii) comparing the level determined in step ii) with a reference value and iv) concluding that the subject suffers from an Familial Mediterranean Fever when the IL-18 level determined at step ii) is higher than the reference value.

The present invention also relates to an in vitro method for diagnosing Familial Mediterranean Fever (FMF) disease in a subject, comprising the steps of i) treating immune primary cells obtained from the subject, with Protein Kinase C (PKC) inhibitors ii) detecting at the time t, t being less than or equal to 3 hours after the treatment of step i), the cell death level of immune primary cells iii) comparing the level determined in step ii) with a reference value and iv) concluding that the subject suffers from an Familial Mediterranean Fever when cell death level determined at step ii) is higher than the reference value.

In a final aspect, the invention relates to a kit comprising means for detecting cytokine selected from the group consisting of IL-18 and or of IL1 beta on a cell population and PKC inhibitors.

### DETAILED DESCRIPTION OF THE INVENTION

Diagnostic Methods According to the Invention

The present invention relates to an in vitro method for diagnosing Familial Mediterranean Fever (FMF) disease in a subject, comprising the steps of i) treating immune primary cells obtained from the subject and beforehand treated with a NF-κB activator, with a Protein Kinase C (PKC) inhibitor ii) detecting the level of IL1 beta secreted from these cells supernatant iii) comparing the level determined in step ii) with a reference value and iv) concluding that the subject suffers from an Familial Mediterranean Fever when the IL1 beta level determined at step ii) is higher than the reference value.

The present invention relates to an in vitro method for diagnosing Familial Mediterranean Fever (FMF) disease in a subject, comprising the steps of i) treating immune primary cells obtained from the subject, with a Protein Kinase C (PKC) inhibitor ii) detecting the level of IL-18 from these cells supernatant iii) comparing the level determined in step ii) with a reference value and iv) concluding that the subject suffers from an Familial Mediterranean Fever when the IL-18 level determined at step ii) is higher than the reference value.

The present invention also relates to an in vitro method for diagnosing Familial Mediterranean Fever (FMF) disease in a subject, comprising the steps of i) treating immune primary cells obtained from the subject, with a Protein Kinase C (PKC) inhibitor ii) detecting at the time t, t being less than or equal to 3 hours after the treatment of step i) the cell death level of immune primary cells iii) comparing the level determined in step ii) with a reference value and iv) concluding that the subject suffers from an Familial Mediterranean Fever when cell death level determined at step ii) is higher than the reference value.

In a preferred embodiment, the time t for detecting the cell death level of immune primary cells is less than or equal to 2 hours after the treatment of step i), in a most preferred embodiment, the time is less than or equal to 1 hour after the treatment of step i).

In a particular embodiment of the second and third in vitro method for diagnosing Familial FMF (with IL-18 biomarker and cell death biomarker), the immune primary cells are previously to step i) treated with NF-κB activator.

As used herein the term “NF-κB” or “NF-κappaB” or “nuclear factor kappa-light-chain-enhancer of activated B cells” designates a protein complex that controls transcription of DNA, cytokine production and cell survival. NF-κB is found in almost all animal cell types and is involved in cellular responses to stimuli such as stress, cytokines, free radicals, heavy metals, ultraviolet irradiation, oxidized LDL, and bacterial or viral antigens (Gilmore T D (2006). Oncogene. 25 (51): 6680-4. Perkins N D (January 2007). Nature Reviews Molecular Cell Biology. 8 (1): 49-62. Gilmore T D (November 1999). Oncogene. 18 (49): 6842-4.) NF-κB plays a key role in regulating the immune response to infection.

As used herein the term “NF-κB activator” or “NF-κappaB activator” designates a molecule, which preferably directly activates NF-κappaB. The activator may be of various natures, such as a bacterium, a virus, a protein (such as cytokine, a growth factor, a hormone), a peptide, a lipid (such as LPS) or is a small chemical molecule. Examples of NF-κB activator include but are not limited to any of the NF-κB activator described in Pahl H L. (Oncogene (1999) 18,6853-6866) all of which are herein incorporated by reference.

In one embodiment the NF-κB activator is a toll-like receptors ligand.

In preferred embodiment the NF-κB activator is a lipid such as Lipopolysaccharides (LPS).

As used herein the term “LPS” or “Lipopolysaccharides”, also known as lipoglycans and endotoxins, designates large molecules consisting of a lipid A and a polysaccharide composed of O-antigen, outer core and inner core joined by a covalent bond; they are found in the outer membrane of Gram-negative bacteria, and elicit strong immune responses in animals.

As used herein the term “Familial Mediterranean Fever” or “FMF” refers to the most frequent hereditary systemic autoinflammatory disorder characterized by short and recurrent episodes of fever and chest/abdominal pain (Sonmez, Batu, and Ozen 2016). Its main complication is secondary amyloidosis, which can lead to renal failure. Its prevalence is highly variable worldwide varying from 1:150 to 1:10,000. In several countries including Turkey, Italy, Israel, Armenia and Japan, FMF is not considered a rare disease (based on the European definition of a prevalence<1:2,000) (Migita et al. 2016; La Regina et al. 2003; Ozen et al. 1998; Ben-Chetrit and Touitou 2009; Daniels et al. 1995; Sarkisian et al. 2008). Colchicine is the main treatment for FMF and is highly efficient in most patients to prevent acute inflammation and amyloidosis by decreasing chronic subclinical inflammation (Goldfinger 1972). Daily and lifelong administration of colchicine is currently recommended for FMF patients.

As used herein the term “PKC” or “Protein kinase C” (EC 2.7.11.13), is a family of protein kinase enzymes that are involved in controlling the function of other proteins through the phosphorylation of hydroxyl groups of serine and threonine amino acid residues on these proteins, or a member of this family. PKC enzymes in turn are activated by signals such as increases in the concentration of diacylglycerol (DAG) or calcium ions (Ca²⁺) (Wilson C H, et al (2015). The Biochemical Journal. 466 (2): 379-90). Hence PKC enzymes play important roles in several signal transduction cascades (Ali E S, et al (2016) “BBA-Molecular Cell Research. 1863: 2135-46)

The PKC superfamily consists of fifteen isozymes in humans (Mellor H, Parker P J (1998). The Biochemical Journal. 332. 332 (Pt 2): 281-92). They are divided into three subfamilies, based on their second messenger requirements: conventional (or classical), novel, and atypical (Nishizuka Y (1995). FASEB Journal. 9 (7): 484-96). Conventional (c) PKCs contain the isoforms α, βI, βII, and γ. These require Ca²⁺, DAG, and a phospholipid such as phosphatidylserine for activation. Novel (n) PKCs include the δ, ε, η, and θ isoforms, and require DAG, but do not require Ca²⁺ for activation. Thus, conventional and novel PKCs are activated through the same signal transduction pathway as phospholipase C. On the other hand, atypical (a) PKCs (including protein kinase Mζ and ι/λ iso forms) require neither Ca²⁺ nor diacylglycerol for activation. Finally, the most recently discovered PRKs define a fourth grouping consisting of at least three members, PRKs 1-3. PRK1 (PKN) was isolated in PCR-based and low-stringency screening, (Hashimoto T, et al. Brain Res. Mol Brain Res. 1998; 59 (2):143-53; Quilliam L A, et al. J. Biol Chem. 1996; 271(46):28772-6). Like the aPKCs, PRKs are insensitive to Ca²⁺, DAG and phorbol esters (Volonte C, et al. J. Biol. Chem. 1992; 267 (30):21663-70. Manser C, et al. FEBS Lett. 2008; 582 (15):2303-8). However, PRK1 has been shown to bind to the activated RhoA GTPase, which leads to a 4-fold activation of the kinase in vitro (Xiao S, et al. Biochim. et Biophys. Act. 2006; 1762 (11-12):1001-12). It has also been shown that the other fully cloned member of the PRK subfamily, PRK2, is also capable of binding RhoA (Taniguchi T, et al. J. Biol Chem. 2001; 276 (13):10025-31; Tomaselli B, et al. Am. J Biochem Biotechnol. 2005; 2(3):161-167), suggesting that this is a general property of this group

The term “protein kinase C” and “protein kinase C superfamily” usually refers to the entire family of isoforms.

As used herein the term “PKC inhibitors” refers to any PKC superfamily inhibitor that is currently known in the art or that will be identified in the future, and includes any chemical entity that, upon administration to a patient, results in inhibition of a biological activity associated with activation of the PKC, including any of the downstream biological effects otherwise resulting from the activation of the PKC. Such PKC inhibitor include any agent (chemical entity, antibody . . . ) that may block PKC activation or any of the downstream biological effects of PKC activation. Such an inhibitor may act by binding directly to the enzyme and inhibiting its kinase activity.

Examples of PKC inhibitor include but are not limited to
Enzastaurin (LY317615) (CAS No. 170364-57-5): is a potent PKCβ selective inhibitor with IC50 of 6 nM in cell-free assays, 6- to 20-fold selectivity against PKCα, PKCγ and PKCε. (Phase 3).
Sotrastaurin (CAS No. 425637-18-9): is a potent and selective pan-PKC inhibitor, mostly for PKCθ with Ki of 0.22 nM in a cell-free assay; inactive to PKCζ. (Phase 2).
Staurosporine (CAS No. 62996-74-1): is a potent PKC inhibitor for PKCα, PKCγ and PKC_{η} with IC50 of 2 nM, 5 nM and 4 nM, less potent to PKCδ (20 nM), PKCε (73 nM) and little active to PKCζ (1086 nM) in cell-free assays. Also shows inhibitory activities on other kinases, such as PKA, PKG, S6K, CaMKII, etc. (Phase 3).
UCN-01 is a synthetic derivative of staurosporine (CAS No. 112953-11-4) also called 7-hydroxystaurosporine is a selective inhibitor of PKC (IC50=29 nM, 34 nM, 30 nM, 590 nM and 530 nM for PKCα, PKCβ, PKCγ, PKCδ and PKCε)
Go 6983 (CAS No. 133053-19-7): is a pan-PKC inhibitor against for PKCα, PKCβ, PKCγ and PKCδ with IC50 of 7 nM, 7 nM, 6 nM and 10 nM, respectively; less potent to PKCζ and inactive to PKCμ.
Bisindolylmaleimide I (GF109203X) (CAS No. 133052-90-1): is a potent PKC inhibitor with IC50 of 20 nM, 17 nM, 16 nM, and 20 nM for PKCα, PKCβI, PKCβII, and PKCγ in cell-free assays, respectively, showing more than 3000-fold selectivity for PKC as compared to EGFR, PDGFR and insulin receptor.
LY333531 HCl (Ruboxistaurin) (CAS No. 169939-93-9): a β-specific protein kinase C inhibitor. It competitively and reversibly inhibits PKCβ1 and PKCβ2 with IC50 values of 4.7 and 5.9 nM respectively.
Ro 31-8220 Mesylate (CAS No. 138489-18-6): a pan-PKC inhibitor with IC50 of 5 nM, 24 nM, 14 nM, 27 nM, and 24 nM for PKC-α, PKC-βI, PKC-βII, PKC-γ, and PKC-ε, respectively.
Daphnetin (CAS No. 486-35-1): a natural coumarin derivative, is a protein kinase inhibitor, inhibits EGFR, PKA and PKC with IC50 of 7.67 μM, 9.33 μM and 25.01 μM, respectively, also known to exhibit anti-inflammatory and anti-oxidant activities.
Dequalinium Chloride (CAS No. 522-51-0): is a PKC inhibitor with IC50 of 7-18 μM, and also a selective blocker of apamin-sensitive K+ channels with IC50 of 1.1 μM.
Quercetin (CAS No. 117-39-5): a natural flavonoid present in vegetables, fruit and wine, is a stimulator of recombinant SIRT1 and also a PI3K inhibitor with IC50 of 2.4-5.4 μM. (Phase 4)
Myricitrin (CAS No. 17912-87-7): a flavonoid compound isolated from the root bark of Myrica cerifera, which exerts antinociceptive effects.
Go6976 (CAS No. 136194-77-9): is a potent PKC inhibitor with IC50 of 7.9 nM, 2.3 nM, and 6.2 nM for PKC (Rat brain), PKCα, and PKCβ1, respectively.
Midostaurin (PKC412) (CAS No. 120685-11-2: is a multi-targeted kinase inhibitor, including PKCα/β/γ, Syk, Flk-1, Akt, PKA, c-Kit, c-Fgr, c-Src, FLT3, PDFRβ and VEGFR1/2 with IC50 ranging from 80-500 nM

Examples of PKC inhibitor include but are not limited to any of the PKC inhibitor described in Sobhia M E. et al. (Exp Opin. Ther Pat (2013), 23:11) in Lee R M, et al (Exp Opin Ther Targets, (2008) 12:5) all of which are herein incorporated by reference.

In specific embodiment, the PKC inhibitor targets the PKN (PRK) members of the PKC superfamily. In a most specific embodiment, the PKC inhibitor targets the PKN1 (PRK1) and or PKN2 (PRK2) members of the PKC superfamily.

In preferred embodiment, the PKC inhibitor is selected from the group consisting of Staurosporine, UCN-01 and Ro-31-8220.

The term “diagnosis” means the identification of the condition or the assessment of the severity of the disease.

In the context of the present invention the “diagnosis” is associated with level of cytokine selected from the group consisting of IL-18 and IL1 beta and/or level of cell death biomarkers which in turn may be a risk for developing a FMF.

Such methods comprise contacting an immune primary cell sample obtained from the subject to be tested under conditions allowing detection of IL1 beta or IL-18 cytokine and/or cell death. Once the sample from the subject is prepared, the level of inflammasome biomarkers (IL1 beta or IL-18 or cell death) may be measured by any known method in the art.

For example, the level of IL1 beta or IL-18 may be measured by using standard immunodiagnostic techniques using anti-IL1 beta or IL-18 antibody, including immunoassays such as competition, direct reaction, or sandwich type assays. Such assays include, but are not limited to, Western blots, agglutination tests, enzyme-labeled and mediated immunoassays such as ELISA, biotin/avidin type assays, radioimmunoassays, immunoelectrophoresis, immunoprecipitation.

Specifically, the Anti-IL1 beta or IL-18 antibodies are commercially available;
cleaved IL-1b (Asp116) (D3A3Z) rabbit mAb #83186 from Cell Signaling Technology
IL-18 (1.51 E3E1) monoclonal Antibody from Santa Cruz Biotechnology.

In another embodiment, the level of IL1 beta (or IL-18) may also be measured by using standard immunochemical methods (Elisa) in order to detect the level of IL1 beta as described in the Example (Duoset ELISA DY201 from R&D systems).

The level of cell death may be also measured by any known method in the art

For example, the level of cell death by using standard techniques such as propidium iodide (Pierini R, Cell death and differentiation 2012) Colorimetric Assays (i.e. LDH release)

Non-radioactive cytotoxicity assay, Cell Viability Assay (i.e. dye exclusion assay with trypan blue) Electron Microscopy Investigation Of Apoptosis, Assay For Estimation Of DNA Fragmentation (i.e; Tunel assay), Caspase Activity Assays, Annexin V Assay, Analyses Of Complex Mitochondria Function During Apoptosis.

Examples of such standard techniques to assay cell death level are described in Juricic V. et al. (Arch Oncol 2008; 16 (3-4):49-54) all of which are herein incorporated by reference

Typically, the high or low level of cytokine biomarker (IL1 beta or IL-18) or cell death biomarker is intended by comparison to a control reference value.

Said reference control values may be determined in regard to the level of cytokine or cell death biomarker present in blood samples taken from one or more healthy subject(s) or to the cytokine biomarker or cell death distribution in a control population.

In one embodiment, the method according to the present invention comprises the step of comparing said level of cytokine biomarker (IL1 beta or IL-18) to a control reference value wherein a high level of cytokine biomarker (IL-1 beta or IL-18) or cell death biomarker compared to said control reference value is predictive of a high risk of having a FMF and a low level of cytokine biomarker (IL1 beta or IL-18) or cell death biomarker compared to said control reference value is predictive of a low risk of having a FMF.

The control reference value may depend on various parameters such as the method used to measure the level of cytokine biomarker (IL1 beta or IL-18) or cell death biomarker or the gender of the subject.

Typically regarding the reference value using cytokine biomaker, as indicated in the Example section (FIG. 1A), for a level of IL-1 beta in monocyte supernatant measured using an immunoassay with an antibody raised against human IL-1 beta, a level of IL-1 beta superior to 50 pg/ml, is predictive of having or a high risk of having a FMF and a level of IL-1 beta lower than 50 pg/ml is predictive of not having a low risk of having a FMF.

Typically regarding the reference value using cell death biomarker, as indicated in the Example section (FIG. 5) for a cell death level of monocyte measured using a PI assay after activation with staurosporin, a level of monocyte cell death superior to 10%, is predictive of having or a high risk of having a FMF and a level of monocyte cell death lower than 10% is predictive of a low risk of having a FMF.

Control reference values are easily determinable by the one skilled in the art, by using the same techniques as for determining the level of cytokine biomarker or cell death in blood samples previously collected from the patient under testing.

A “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the cytokine (IL-1 beta or IL-18) level or cell death level (obtained according to the method of the invention) with a defined threshold value. In one embodiment of the present invention, the threshold value is derived from the activated monocyte level (or ratio, or score) determined in a blood sample derived from one or more subjects who are responders (to the method according to the invention). In one embodiment of the present invention, the threshold value may also be derived from immune primary cells activated with PKC inhibitor (or ratio, or score) determined in a blood sample derived from one or more subjects or who are non-responders. Furthermore, retrospective measurement of the activated immune primary cells level (or ratio, or scores) in properly banked historical subject samples may be used in establishing these threshold values. In a preferred embodiment of the present invention, the threshold value may be determined using an immune primary cells sample derived from the same subject without stimulation (internal control).

Reference values are easily determinable by the one skilled in the art, by using the same techniques as for determining the level of activated monocytes in fluids samples previously collected from the patient under testing.

“Risk” in the context of the present invention, relates to the probability that an event will occur over a specific time period, as in the conversion to Familial Mediterranean fever (FMF), and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no conversion. Alternative continuous measures, which may be assessed in the context of the present invention, include time to FMF conversion risk reduction ratios.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to a FMF condition or to one at risk of developing a FMF. Risk evaluation can also comprise prediction of future clinical parameters, traditional laboratory risk factor values, or other indices of FMF, such as cellular population determination in peripheral tissues, in serum or other fluid, either in absolute or relative terms in reference to a previously measured population. The methods of the present invention may be used to make continuous or categorical measurements of the risk of conversion to FMF, thus diagnosing and defining the risk spectrum of a category of subjects defined as being at risk for a FMF. In the categorical scenario, the invention can be used to discriminate between normal and other subject cohorts at higher risk for FMF. In other embodiments, the present invention may be used so as to help to discriminate those having FMF from normal.

The term “immune primary cell” has its general meaning in the art and is intended to describe a population of white blood cells directly obtained from a subject.

In the context of the present invention immune primary cell is selected from the group consisting of PBMC, WBC, monocyte or neutrophil.

The term “PBMC” or “peripheral blood mononuclear cells” or “unfractionated PBMC”, as used herein, refers to whole PBMC, i.e. to a population of white blood cells having a round nucleus, which has not been enriched for a given sub-population (which contain monocytes, T cells, B cells, natural killer (NK) cells, NK T cells and DC precursors). A PBMC sample according to the invention therefore contains lymphocytes (B cells, T cells, NK cells, NKT cells) and monocytes. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer, which will preferentially lyse red blood cells. Such procedures are known to the expert in the art.

The term “WBC” or “White Blood Cells”, as used herein, also refers to leukocytes population, are the cells of the immune system. All white blood cells are produced and derived from multipotent cells in the bone marrow known as hematopoietic stem cells. Leukocytes are found throughout the body, including the blood and lymphatic system. Typically, WBC or some cells among WBC can be extracted from whole blood by using i) immunomagnetic separation procedures, ii) percoll or ficoll density gradient centrifugation, iii) cell sorting using flow cytometer (FACS). Additionally, WBC can be extracted from whole blood using a hypotonic lysis buffer, which will preferentially lyse red blood cells. Such procedures are known to the expert in the art.

In some embodiments, the fluid sample is a sample of purified monocyte or neutrophil in suspension. Typically, the sample of monocyte or neutrophil is prepared by immunomagnetic separation methods preformed on a PBMC or WBC sample. For example, monocytes cells are isolated by using antibodies for monocytes —associated cell surface markers, such as CD14. Commercial kits, e.g. MACS cell separation kits using CD14 microbeads, human (#130-050-201 from Miltenyl Biotec) are available.

Kits of the Invention:

A further object of the invention relates to kit for diagnosing Familial Mediterranean Fever (FMF) comprising means for detecting of IL1 beta or IL-18 (or cell death) on a cell population and a PKC inhibitor.

In some embodiments, said means for detecting cell death are antibodies. In another embodiment, these antibodies are labelled as above described.

Typically, the kits described above will also comprise one or more other containers, containing for example, wash reagents, and/or other reagents capable of quantitatively detecting the presence of bound antibodies. Preferably, the detection reagents include labelled (secondary) antibodies or, where the antibody raised against IL1 beta is itself labelled, the compartments comprise antibody binding reagents capable of reacting with the labelled antibody. A compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the test sample, a container which contains the antibody(s) used in the assay, containers which contain wash reagents (such as phosphate buffered saline, Tris-buffers, and like), and containers which contain the detection reagent.

Typically, a kit of the present invention will also include instructions for using the kit components to conduct the appropriate methods.

Therapeutic Method

As mentioned Colchicine is the main treatment for FMF and is highly efficient in most patients to prevent acute inflammation and amyloidosis by decreasing chronic subclinical inflammation (Goldfinger 1972). Daily and lifelong administration of colchicine is currently recommended for FMF patients. Other current treatment for FMF are IL1 antagonist such as Canakinumab (trade name Ilaris, previously ACZ885) [2] a human monoclonal antibody targeted at interleukin-1 beta (Dhimolea, Eugen (2010). MAbs. 2 (1): 3-13) and Anakinra (brand name Kineret) which is an interleukin 1 (IL1) receptor antagonist; a small molecule used to treat rheumatoid arthritis (Fleischmann R M, et al. (2006). Annals of the rheumatic diseases. 65 (8): 1006-12 1)

The invention also relates to a method for treating a Familial Mediterranean fever (FMF) patient with colchicine or IL1 antagonist in a subject wherein the level of cytokine (IL-18 or IL1 beta) secreted by immune primary cells (or cell death level of these cells) obtained from said patient, which have been beforehand treated with a Protein Kinase C (PKC) inhibitor, have been detected by one of method of the invention.

Another object of the present invention is a method of treating Familial Mediterranean fever (FMF) in a subject comprising the steps of:

a) providing immune primary cells from a subject,

b) treating these immune primary cells and beforehand treated with a NF-κB activator, with a Protein Kinase C (PKC) superfamily inhibitor ii) detecting the level of IL1 beta secreted from these cells supernatant iii) comparing the level determined in step ii) with a reference value

and

if the IL1 beta level determined at step ii) is higher than the reference value, treating the subject with colchicine or IL1 antagonist.

The invention will be further illustrated by the following figures and examples. However, these examples and figures should not be interpreted in any way as limiting the scope of the present invention.

### FIGURES

FIG. 1: Monocytes from FMF Patients Specifically Secrete Higher Levels of IL-1β than Monocytes from Healthy Donors in Response to Various PKC Superfamily Inhibitors.

Primary monocytes (5×10³ per well of a 96 well plates) from HD or FMF patients presenting 2 clearly pathogenic variants (CPV) were primed with LPS (10 ng/ml) during 3 h and stimulated with (A) staurosporin (1.25 μM), (B) UCN-01 (12.5 μM), (C) Ro-31-8820 (100 μM), (D) ATP (2.5 mM) for 1.5 h. IL-1β levels in the supernatant were determined by ELISA. Each symbol represents the average value from three technical replicates per HD (n=3 to 22 as indicated) or FMF patients (n=4 to 26 as indicated). The bar represents the mean.

FIG. 2: Monocytes from FMF Patients Die Faster than Monocytes from Healthy Donors in Response to a PKC Superfamily Inhibitor but not in Response to LPS+Nigericin Treatment.

Primary monocytes (2×10⁴ per well of a 96 well plates) from HD or FMF patients presenting 2 clearly pathogenic variants (CPV) were stimulated with (A-C) UCN-01 (12.5 μM) or (D-G) LPS 10 ng/ml during 3 h followed by nigericin (5 μM) in the presence of propidium iodide. Propidium iodide incorporation was monitored every 5 minutes from 5 minutes to 105 minutes post-UCN-01/nigericin stimulation using a fluorimeter. Cell death was calculated using TX-100-treated cells (100% cell death) and normalized cell death kinetics are presented (A, D). Each symbol corresponds to the average values of the normalized cell death of monocytes from 17 HD and 11 FMF patients. Mean and standard deviations are shown at each time point. (B, E, F) The Area Under the Curve (AUC) was calculated for each HD (n=17) and FMF patients (n=13) cell death kinetics from 15 to 60 min (B, E) or to 105 min (F) post-UCN-01 (B) or post-nigericin (E-F) treatment. (C-G) The time post-treatment needed to reach 20% monocytes cell death was calculated from each HD (n=17) and FMF patients (n=13) cell death kinetics. (B-C, E-G) Each symbol represents the mean value from three technical replicates per HD (n=17) or FMF patients (n=13). The bar represents the mean.

FIG. 3: Monocytes from Patients Suffering from Inflammatory Diseases Other than FMF do not Hyper-Respond PKC Superfamily Inhibitors.

Primary monocytes from HD or patients suffering from various inflammatory or infectious diseases (ID) (CAPS (n=1), HIDS (n=1), Behcet's disease (n=4), lupus (n=3), Still's disease (n=4), non-systemic juvenile idiopathic arthritis (n=1), sepsis (n=3), inflammatory bowel disease (n=4)) were (A, B) primed with LPS (10 ng/ml) during 3 h and stimulated with (A) staurosporin (1.25 μM), (B) UCN-01 (12.5 μM). (A-B) IL-1β levels in the supernatant were determined by ELISA. Each symbol represents the average value from three technical replicates per HD (n=16 to 22 as indicated) or ID patients (n=18). The bar represents the mean. (C-H) monocytes were stimulated with (C, E, G) UCN-01 (12.5 μM) or (D, F, H) LPS 10 ng/ml during 3 h followed by nigericin (5 μM) in the presence of propidium iodide. Propidium iodide incorporation was monitored every 5 minutes from 5 minutes to 105 minutes post-UCN-01/nigericin stimulation using a fluorimeter. Cell death was calculated using TX-100-treated cells (100% cell death) and normalized cell death kinetics are presented (C, D). Each symbol corresponds to the average values of the normalized cell death of monocytes from 17 HD and 18 ID patients. Mean and standard deviations are shown at each time point. (E, F) The Area Under the Curve (AUC) was calculated for each HD (n=17) and ID patients (n=18) cell death kinetics from 15 to 60 min post-UCN-01 (E) or post-nigericin (F) treatment. (G-H) The time post-treatment needed to reach 20% monocytes cell death was calculated from each HD (n=17) and ID patients (n=18) cell death kinetics. (E-H) Each symbol represents the mean value from three technical replicates per HD (n=17) or ID patients (n=18). The bar represents the mean.

FIG. 4: Monocytes from FMF Patients Presenting One Clearly Pathogenic MEFV Variant Display Heterogeneous Responses to PKC Inhibitors.

Primary monocytes from HD or FMF patients displaying a single clearly pathogenic MEFV variant (CPV) were (A) primed with LPS (10 ng/ml) during 3 h and stimulated with (A) staurosporin (1.25 μM), or directly treated with (B, C) UCN-01 (12.5 μM). (A) IL-1β levels in the supernatant were determined by ELISA. Each symbol represents the average value from three technical replicates per HD (n=22) or FMF patients (n=6). The bar represents the mean. The uppermost dotted line represents the average value of monocytes from FMF patients bearing two CPV. (B-C) monocytes from HD or FMF patients with the indicated genotypes were stimulated with UCN-01 (12.5 μM) in the presence of propidium iodide. Propidium iodide incorporation was monitored every 5 minutes from 5 minutes to 105 minutes post-UCN-01 stimulation using a fluorimeter. Cell death was calculated using TX-100-treated cells (100% cell death) and normalized cell death kinetics are presented (B-C). Two independent experiments are shown. The average values (mean) and the standard errors of three technical replicates from one individual are shown.

FIG. 5: Monocytes from FMF Patients Die More than Monocytes from Healthy Donors in Response to a 1 h Stimulation with a PKC Superfamily Inhibitor.

Primary monocytes from HD or FMF patients were primed with LPS (10 ng/ml) during 3 h and stimulated with staurosporin (1.25 μM). Cell death was determined by measuring propidium iodide incorporation/fluorescence at 1 h30 post stauroporin addition. Each symbol represents the average value from three technical replicates per HD (n=9) or FMF patients (n=7). The bar represents the mean. Cell death was normalized using TX-100-treated cells (100% cell death). With a cell death threshold set at 10% (horizontal line), this assay discriminates healthy donors from FMF patients.

FIG. 6: PKC Inhibitors-Mediated Inflammasome Activation Discriminates FMF Patients from Patients Suffering from Unrelated Inflammatory Conditions.

Receiver Operating Characteristic (ROC) curves were computed for IL-1β concentrations following (F) staurosporin or (G) UCN-01 treatment, (H) the time to obtain 20% cell death and (I) the area under the cell death kinetics curve. For each ROC curve, the AUC, the positive (ppv) and negative (npv) predictive values are indicated.

### EXAMPLE

Methods:

Subjects

Twenty-six patients with FMF were included along with patients suffering from inflammatory diseases from other aetiologies (CAPS (n=1), HIDS (n=1), Behcet's disease (n=4), lupus (n=3), Still's disease (n=4), non-systemic juvenile idiopathic arthritis (n=1), sepsis (n=3), inflammatory bowel disease (n=4)) and 26 HD. All FMF patients fulfilled the Tel Hashomer criteria for FMF and had at least one mutation in the MEFV gene. The potential carriage of MEFV mutation in HD was not assessed. Blood samples from HD were drawn on the same day as patients.

Ethic Statement

The study was approved by the French Comité de Protection des Personnes (CPP, # L16-189) and by the French Comité Consultatif sur le Traitement de l'Information en matière de Recherche dans le domaine de la Santé (CCTIRS, #16.864). The authors observed a strict accordance to the Helsinki Declaration guidelines and informed written consent have been obtained from every patient or its legal representative. HD blood was provided by the Etablissement Francais du Sang in the framework of the convention #14-1820.

Monocyte Isolation

Blood was drawn in heparin-coated tubes and kept at room temperature overnight. The next day, peripheral blood mononuclear cells (PBMCs) were isolated by density-gradient centrifugation using Lymphocyte Separation Medium (Eurobio) [23]. Monocytes were isolated from PBMCs by magnetic selection using CD14 MicroBeads (Miltenyi Biotec) [24] and the AutoMACS Pro Separator (Miltenyi Biotec) following manufacturer's instructions. Monocytes were enumerated in the presence of a viability marker (propidium iodide, 10 μg/ml) by flow cytometry (BD Accuri C6 Flow Cytometer®) [25].

Inflammasome Activation

Monocytes were seeded in 96-well plates at 5×103 cells/well in RPMI 1640, GlutaMAX medium (Thermofisher) supplemented with 10% fetal calf serum (Lonza). When indicated, monocytes were incubated for 3 hours in the presence of LPS (10 ng/ml, Invivogen). Unless otherwise indicated, cells were then treated for 1 h30 with nigericin (5 μM, Invivogen) and ATP (2.5 mM, Sigma) [27]; staurosporin (1.25 μM; Tocris); UCN-01 (12.5 μM; Tocris) or Ro31-8820 (100 μM; Tocris). Of note, Staurosporin from other vendors displayed 10-fold lower activity than the one we used. Following the incubation, cells were centrifuged and supernatants were collected.

Cytokine Detection and Cell Death Assay

Levels of IL-1β in monocyte supernatants were quantified by ELISA (R&D Systems). Cell death was monitored by incubating 2×104 monocytes per well of a black 96 well plate (Costar, Corning) with propidium iodide (PI, Sigma) at 5 μg/ml. Three technical replicates per conditions were done. UCN-01 was added at 12.5 μM in the absence of any priming signal. Nigericin was added at 5 μM after a 3 h priming with LPS at 10 ng/ml. Real time PI incorporation was measured every 5 minutes from 15 minutes to 105 inutes post-Nigericin/UCN-01 intoxication on a fluorimeter (Tecan) using the following wavelengths: excitation 535 nm (bandwidth 15 nm); emission 635 nm (bandwidth 15 nm) [29,30]. Cell death was normalized using PI incorporation in monocytes treated with triton X100 for 15 min (=100% cell death) and PI incorporation at each time point in untreated monocytes (0% cell death). As a further correction, the first time point of the kinetics was set to 0. The areas under the curve were computed using the trapezoid rule (Prism 6; GraphPad). To extract the time needed to reach 20% cell death, a non-linear regression analysis (Prism 6; GraphPad) was used to fit a sixth order polynomial curve to the normalized cell death kinetics using the least squares fit as the fitting method. The obtained curve was used to interpolate the time corresponding to 20% cell death.

Result

PKC Superfamily Inhibitors Trigger IL-1β Release Specifically in Monocytes from FMF Patients

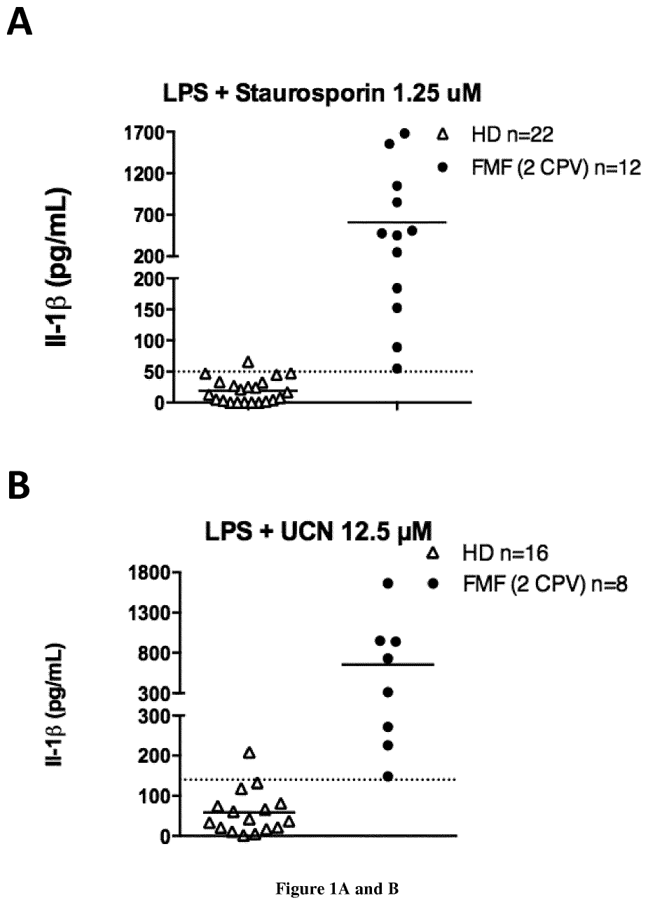

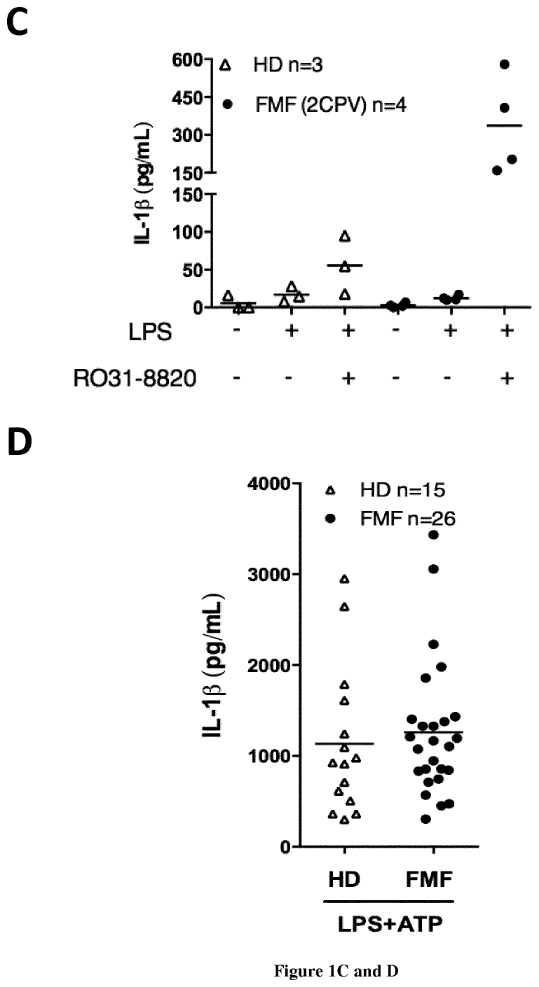

We previously observed that the Pyrin inflammasome was hyper-activated in monocytes from FMF patients compared to HD in response to low doses of the bacterial toxin TcdB (Jamilloux et al. under revision). The difference in the level of IL-1β release or cell death kinetics released by monocytes from FMF patients compared to monocytes from HD was clear upon inclusion of a substantial number of FMF patients and HD. Yet, due to the inter-individual heterogeneity in the Pyrin inflammasome response, assessing the Pyrin inflammasome responses using low doses of TcdB was not sufficient to discriminate on an individual basis FMF patients from HD. We thus decided to test other Pyrin inflammasome-activating stimuli to assess whether they could allow us to discriminate functionally the monocyte response of HD and FMF patients. We first selected a cohort of FMF patients for whom the initial clinical diagnosis had been confirmed by the identification of clearly pathogenic MEFV variants (Shinar et al. 2012) on the two alleles. Patients included were either homozygous for the p.M694V or the p.M694I variants or compound heterozygous (p.[M694I];[p.V726A], p.[M694V];[p.R761H], p.[M680I];[p.V726A]). The broad specificity PKC inhibitor, staurosporin, has been reported to trigger activation of the Pyrin inflammasome in murine macrophages, likely due to the targeting of the Pyrin-inhibiting kinases PKN1/2 (Park et al. 2016). Surprisingly, in our conditions, optimized to detect signal 1+signal 2 responses (Jamilloux et al. under revision), we observed no to very low IL-1β release from monocytes isolated from HD in response to LPS (10 ng/ml) and staurosporin (1.25 μM) (FIG. 1A and Fig. S1). Indeed, in our experimental conditions, monocytes from 21 out of the 22 HD released less than 50 pg/ml of IL-1β (FIG. 1A). In sharp contrast, monocytes from FMF patients released moderate to high levels of IL-1β leading to an average level 30-fold higher than the average level of healthy control monocytes (FIG. 1A). This result indicated deviating inflammasome responses to staurosporin between FMF patients and HD.

To confirm this result, we used UCN-01, a hydroxylated derivative of staurosporin, which displays a better selectivity for PKC kinases (Tamaoki 1991, 0). Similar findings were observed (FIG. 1B) with monocytes from FMF patients releasing>10-fold higher levels of IL-1β than monocytes from healthy controls. The same trends (FIG. 1C) was observed using the bisindolylmaleimide RO 31-8220, another PKC superfamily inhibitor (Davis et al. 1992). This compound was tested with only on a limited number of patients since the difference in IL-1β responses between monocytes from FMF patients and from healthy donors was lower than the one observed upon LPS+staurosporin stimulation. This lower difference might be due to the low efficacy of RO 31-8220 to inhibit PKN2 (Anastassiadis et al. 2011). As previously described (Jamilloux et al. under revision) (Van Gorp et al. 2016), we did not observe any difference in IL-1β release in response to engagement of the NLRP3 inflammasome by LPS and ATP treatment (FIG. 1D) or of the NLRC4 inflammasome (Jamilloux et al. under revision). Altogether, these results indicate that the quantification of IL-1β release in response to PKC superfamily inhibitors can discriminate monocytes from FMF patients from monocytes from HD.

Cell Death Kinetics Upon PKC Inhibitors Treatment Discriminate FMF Patients' Monocytes from HD Monocytes.

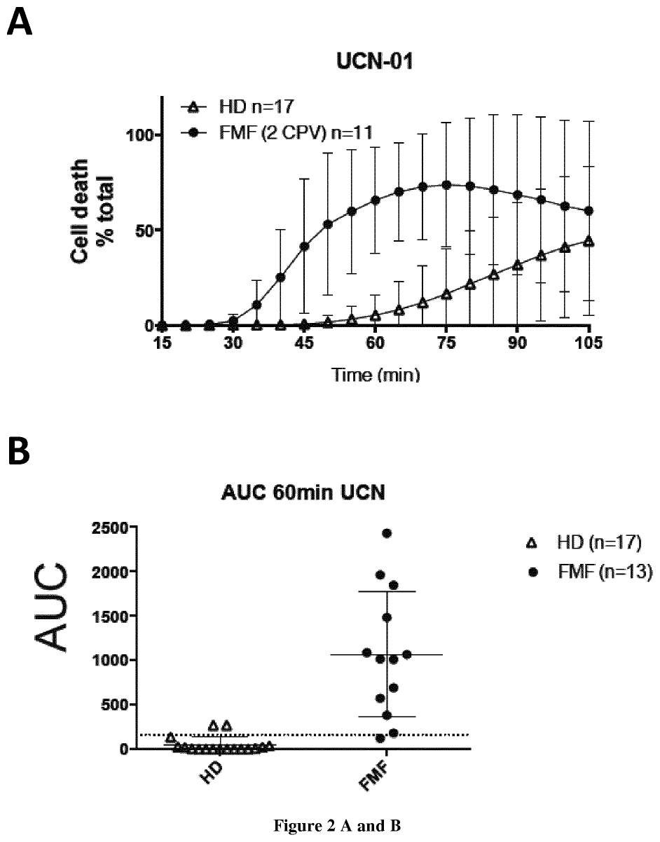

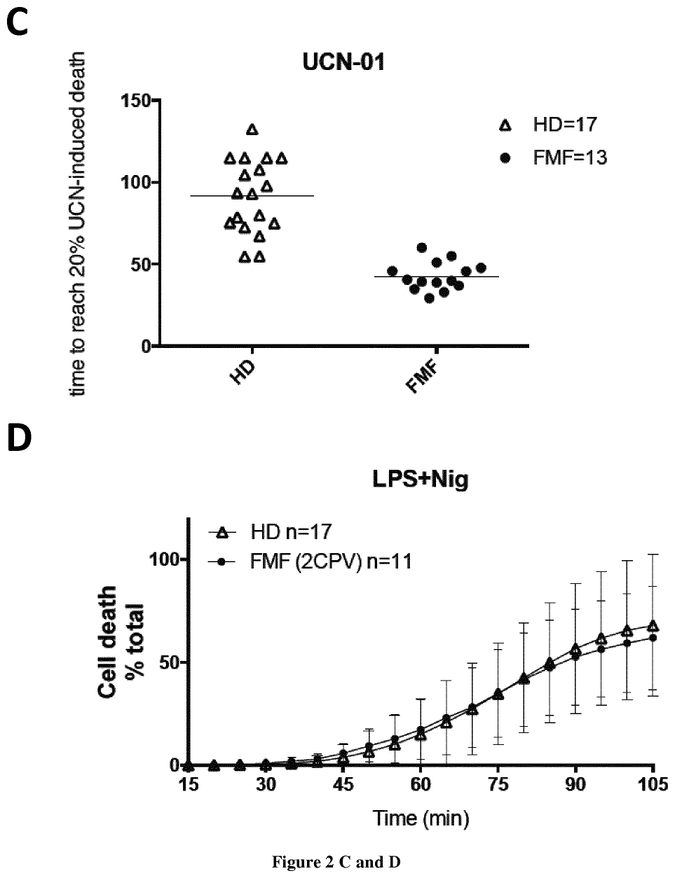

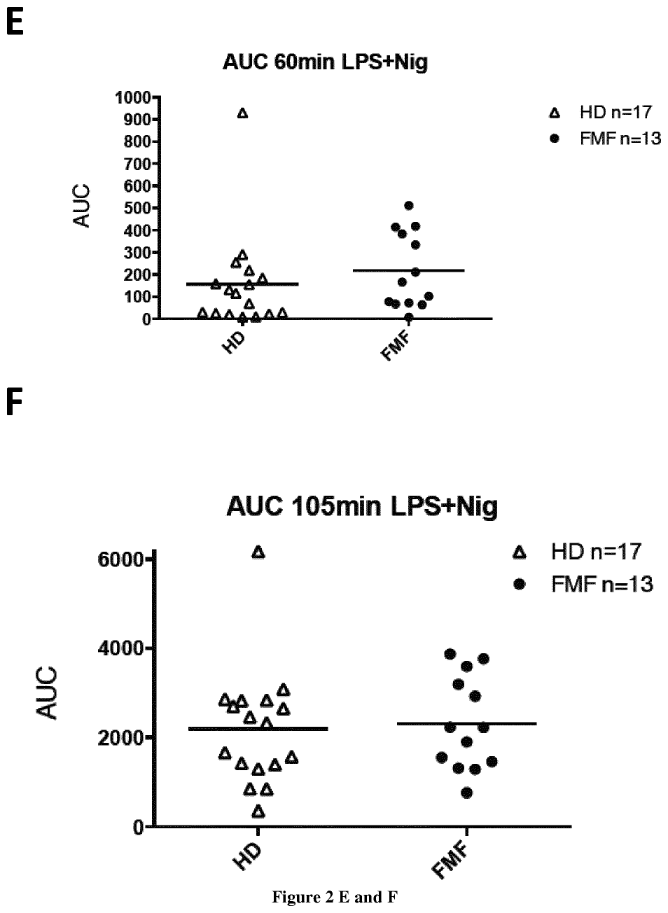

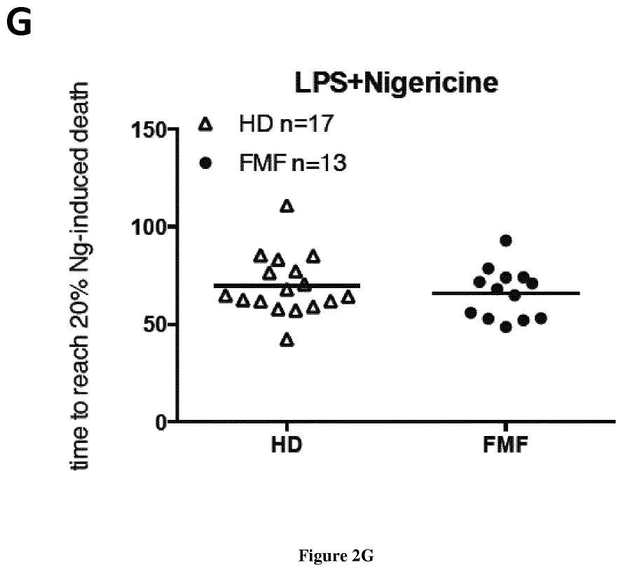

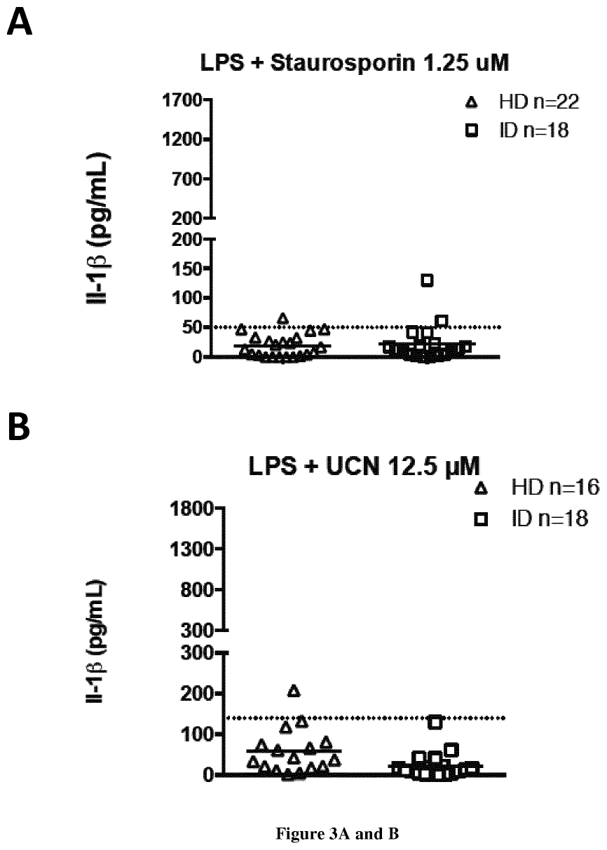

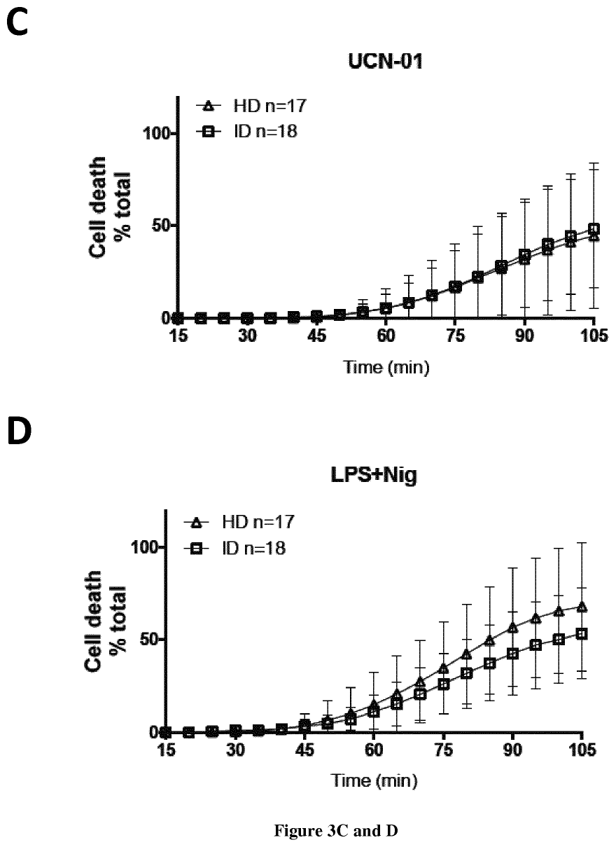

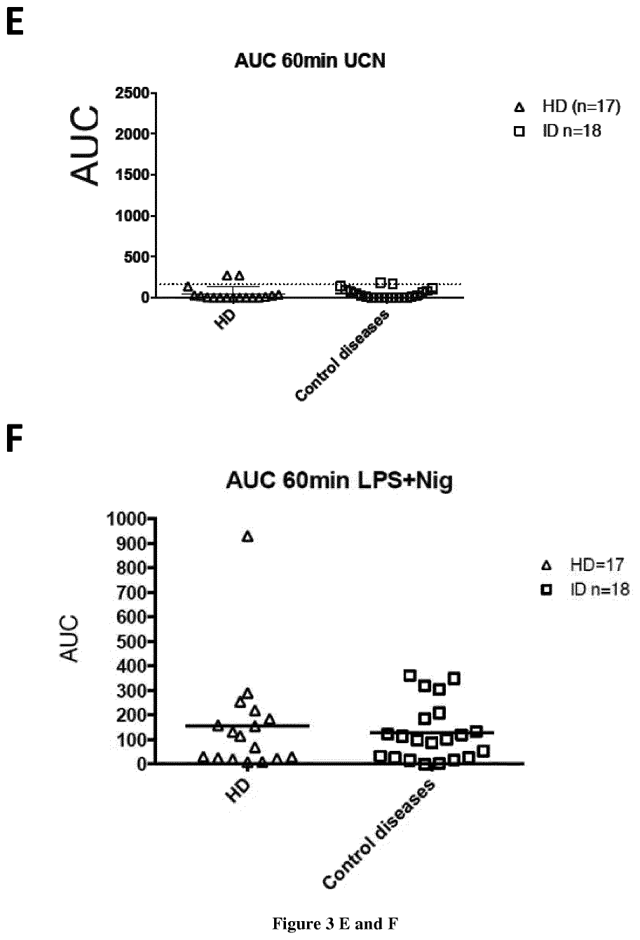

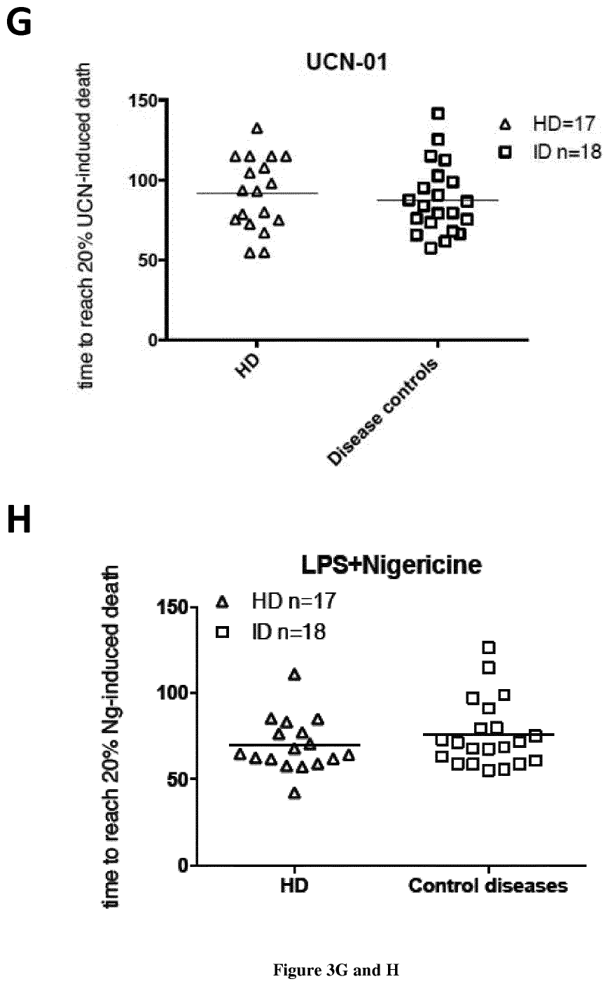

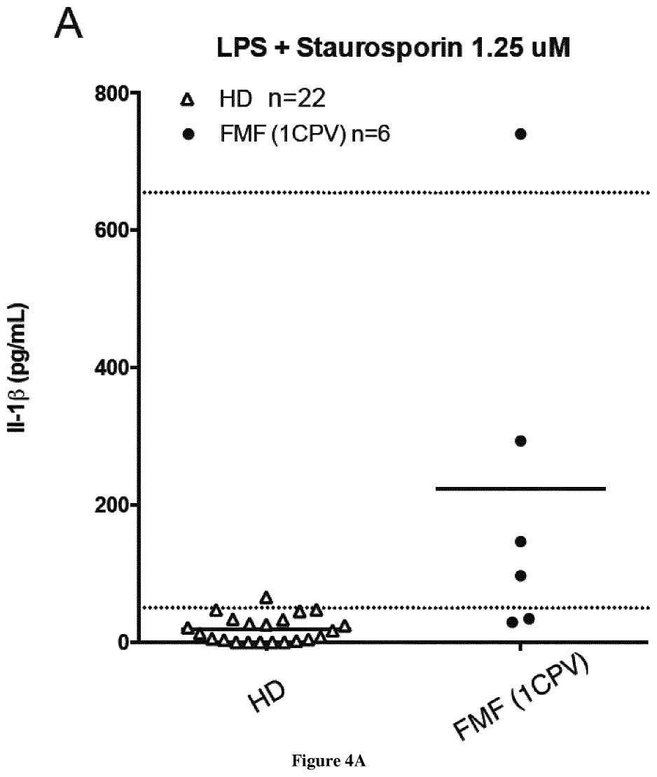

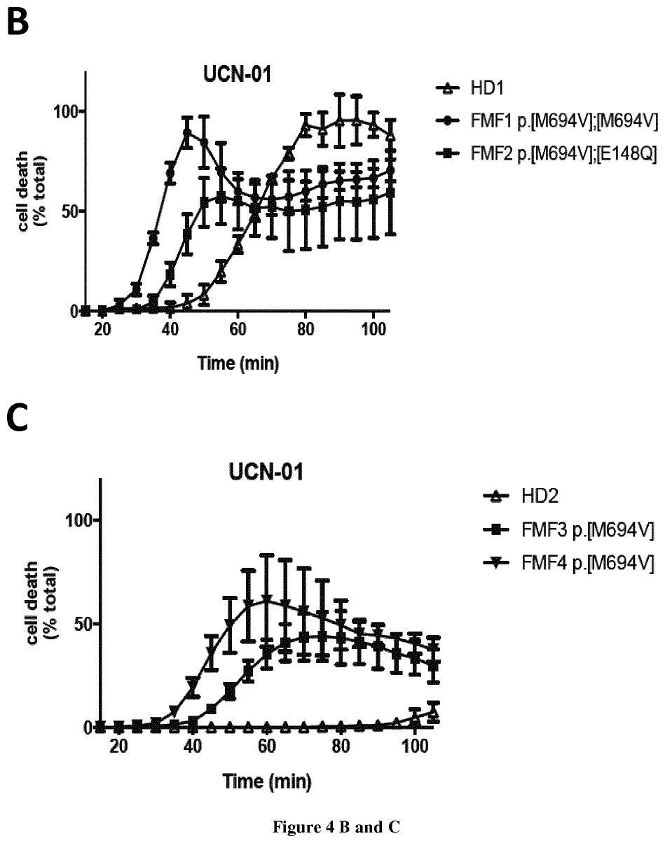

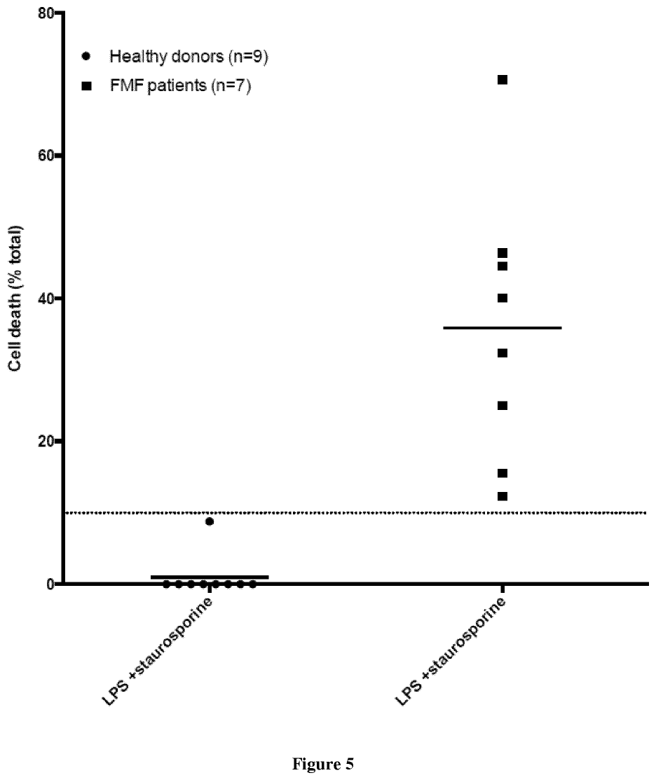

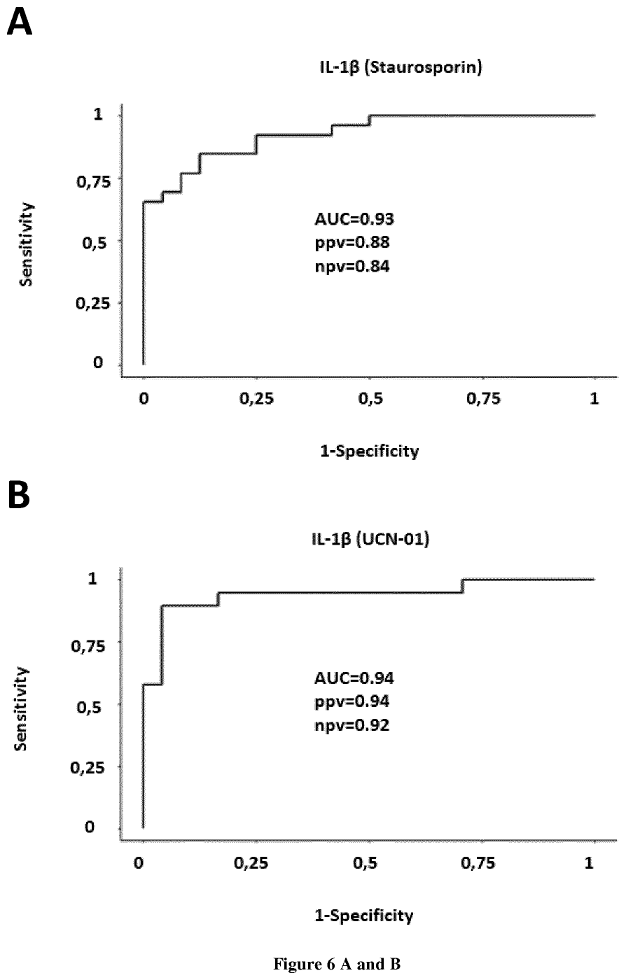

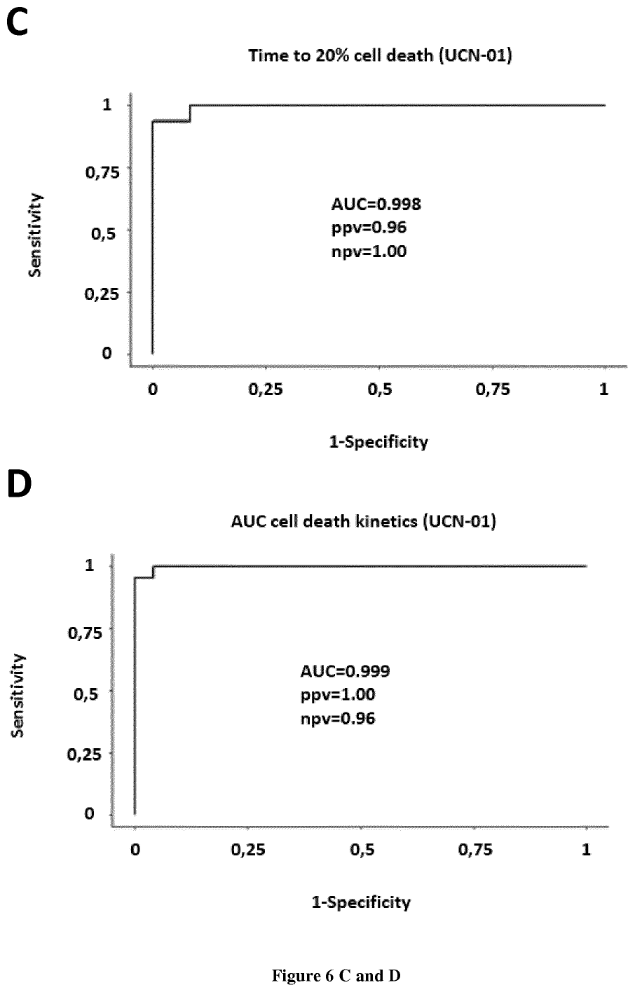

Inflammasome activation leads to the release of the proinflammatory cytokines IL-1β and triggers an inflammatory necrosis termed pyroptosis. While the release of IL-1β requires a priming step to induce the proIL-1β, pyroptosis can be triggered directly upon inflammasome sensor activation. We thus assessed whether the PKC inhibitors in absence of LPS priming would trigger differential pyroptosis kinetics between monocytes from HD and monocytes from FMF patients bearing two clearly pathogenic MEFV variants. We focused on the response to UCN-01, which gave more robust results in a pilot experiment comparing various PKC inhibitors (data not shown). UCN-01 triggered a fast and strong increase in propidium iodide incorporation in the monocytes of FMF patients (FIG. 2A). In contrast, the average response was largely delayed in HD monocytes. Occasionally, we did not even notice any propidium incorporation in the monocytes of healthy donors in response to UCN-01 in the time frame of our experiment (105 min). We calculated the area under the curve (AUC) for each individual kinetics focusing on the first hour of the kinetics and the time required to reach 30% of the maximal cell death as determined using Triton X-100-mediated membrane permeabilisation. The AUC quantification demonstrated a statistical difference in the cell death kinetics of monocytes from FMF patients and healthy controls when exposed to UCN-01 (FIG. 2B). Importantly, this difference was specific for the UCN-01 inhibitor. Indeed, FMF and HD monocytes cell death kinetics overlapped perfectly upon activation of the NLRP3 inflammasome using LPS+nigericin (FIG. 2D). Furthermore, quantification of the cell death kinetics following NLRP3 engagement did not demonstrate any statistical difference in AUC between monocytes from FMF patients and healthy controls (FIG. 2E, F). AUC were calculated with kinetics stopping at either 60 minutes to be comparable with UCN-01 results or 105 minutes to better take into account the nigericin-mediated cell death kinetics. Similarly, quantification of the time needed to reach 20% cell death upon UCN-01 addition demonstrated a statistical difference between monocytes from FMF patients and healthy donors (FIG. 2C). Indeed 20% of FMF patients' monocytes died in less than 50 minutes post-UCN-01 addition while 1 h 30 was needed for monocytes from healthy controls to reach the same cell death level. In contrast, upon LPS+nigericin treatments, the level of 20% cell death was reached in 74 and 72 minutes when using monocytes from FMF patients and from healthy controls, respectively (FIG. 2F). Importantly, these results were also validated using LPS+staurosporin treatment (FIG. 5). Indeed, an average of 35% of monocytes from FMF patients were dead at 1 h 30 post-staurosporin addition while monocytes from none of the healthy donors display more than 10% cell death (set as an arbitrary diagnostic threshold-FIG. 5). These results demonstrate that monocytes from FMF patients are specifically hyper-responsive to PKC superfamily inhibitors, highlighting this functional response as of diagnostic value.

The Hyper-Response to PKC Inhibitors is Specific for Monocytes from FMF Patients.

We then assessed the responses of monocytes from patients presenting a variety of inflammatory diseases, ranging from infectious to autoinflammatory aetiologies. 20 patients were included (CAPS (n=1), HIDS (n=1), Behcet's disease (n=4), lupus (n=3), Still's disease (n=4), non-systemic juvenile idiopathic arthritis (n=1), sepsis (n=3), inflammatory bowel disease (n=4)). Monocytes from these patients were stimulated as described before. Importantly, this analysis clearly demonstrated that the hyper-responsiveness of FMF patients' monocytes to PKC inhibitors was not due to systemic inflammation but rather to a specific defect associated with FMF (FIG. 3 A-H). Of note, monocytes from CAPS patients engage the NLRP3 inflammasome in response to LPS only and reached 30% cell death before addition of the subsequent nigericin stimulus (data not shown). Yet, the kinetics of cell death of CAPS patient monocytes to UCN-01 (which is performed in absence of LPS pretreatment) were similar to the kinetics of healthy controls monocytes further strengthening the specificity of the FMF patients monocytes response to PKC inhibitors.

Monocytes from FMF patients with a single clearly pathogenic MEFV variant demonstrate a heterogeneity of functional responses to PKC inhibitors.

As previously described, genetic confirmation of the FMF clinical diagnosis is frequently impaired by the identification of a single (mono-allelic) clearly pathogenic MEFV variant. We thus decided to assess whether monocytes from patients presenting a clinical FMF diagnosis with only a single clearly pathogenic MEFV variant were hyper-responsive to PKC inhibitors. Monocytes from six FMF patients heterozygous for the p.M694V MEFV variant including one presenting on the second allele the variant of unknown significance p.E148Q were analysed for their IL-1β secretion in response to staurosporin. 4 out of the 6 patients demonstrated IL-1β levels above the 50 pg/ml threshold (FIG. 4A). The average monocyte response from patients displaying a single clearly pathogenic variant (224 pg/ml) was lower than the average monocyte response from patients displaying two clearly pathogenic variants (655 pg/ml-see the uppermost dotted line in FIG. 4A). This difference suggests a gene/variant-dosage effect consistent with the previously reported gene-dosage effects observed at the level of monocyte responses (Omenetti et al. 2014) (Jamilloux Y. et al. under revision) and clinical phenotype (Federici et al. 2012). Yet, monocytes from certain patients carrying a single clearly pathogenic MEFV variant released high IL-1β levels never observed in the supernatant of monocytes from healthy donors even when stimulated with staurosporin doses 10 times higher than the ones used in this assay (FIG. S1). This result indicates that i) the monocytes response of some patients for which genetic testing leads to ambiguous results display a clearly FMF-like signature in terms of IL-1β secretion in response to LPS+stauroporin ii) hyper-responsiveness to staurosporine is complex and not dictated only by the MEFV genotype. The hyper-responsiveness of monocytes from patient with a single clearly pathogenic variant to PKC inhibitors was confirmed using UCN-01 and the real time cell death assay in three patients bearing a single clearly pathogenic MEFV variant (FIG. 4B, 4C). Indeed, monocytes from the three FMF patients bearing a single p.M694V variant died clearly faster than monocytes from healthy controls in response to UCN-01 (FIG. 4B, 4C).

ROC Curve for IL-1 Beta

Due to the strong discrimination between FMF patients and other patients suffering from various conditions with an inflammatory component, we assessed whether the functional response to PKC inhibitors has the potential to be exploited for FMF diagnosis. We thus generated Receiver Operating Characteristic (ROC) curves to determine the sensitivity and specificity of a functional test based on IL-1β dosage following staurosporin (FIG. 6A) or UCN-01 treatment (FIG. 6B) or based on cell death kinetics parameters (time to reach 20% cell death, FIG. 6C or AUC of the cell death kinetics, FIG. 6D). The predictive values (Table 1) and the areas under the ROC curves, which are very close to 1 (1 corresponding to 100% specificity and 100% sensitivity), indicate that these functional assays accurately discriminate FMF patients from other patients presenting inflammatory conditions and from HD.

TABLE 1
Numerical parameters associated with the ROC curves presented in FIG. 6

| Parameter | Inhib. | Threshold | Sens. | Spec. | PPV | NPV | Acc | AUC [Low-Up] |
|---|---|---|---|---|---|---|---|---|
| IL-1β | Staurosporin | 44 | 0.85 | 0.88 | 0.88 | 0.84 | 0.86 | 0.93 [0.86-0.99] |
| IL-1β | UCN-01 | 224 | 0.89 | 0.96 | 0.94 | 0.92 | 0.93 | 0.94 [0.86-1.00] |
| AUC_{RTCD} | UCN-01 | 21 | 0.94 | 1.00 | 1.00 | 0.96 | 0.98 | 0.98 [0.99-1.00] |
| Time_{20% CD} | UCN-01 | 61 | 1.00 | 0.96 | 0.96 | 1.00 | 0.98 | 1.00 [0.99-1.00] |

The threshold values are indicated in pg/mL-1 for IL-1 β, in arbitrary units for the Area Under the real time cell death kinetics curves (AUCRTcD) and minutes for the time to reach 20% cell death (Time_{20% CD}). Sensitivity (Sens.), Specificity (Spec.), Positive Predictive Values (PPV), Negative Predictive Values (NPV), Accuracy (Acc.) for the indicated threshold values are shown. The Area under the ROC curve (AUC) are indicated with their lower and upper values calculated using a 95% confidence interval.

Altogether, our data demonstrate that monocytes from FMF patients display enhanced inflammasome responses to PKC inhibitors paving the way to a functional diagnosis test.

### Discussion

Here, we identified that monocytes from FMF patients are hyper-responsive to PKC superfamily inhibitors. This family includes PKN1/2, two kinases involved in Pyrin inflammasome signalling (Park et al. 2016). While staurosporin, UCN 01 and RO 31-8220 might target other kinases besides PKN1/2, we believe that the effects we observed here on the inflammasome are due to the targeting of PKN1/2 and the dephosphorylation of Pyrin as previously described by others (Park et al. 2016). To our knowledge, there are no inhibitors displaying a strong specificity towards PKN1/2 (Anastassiadis et al. 2011). Furthermore, genetic invalidating of PKN1/2 triggers Pyrin inflammasome activation and cell death impairing a genetic validation of the targeting of these two targets by the inhibitors tested in our assays.

Focusing on monocytes from FMF patients presenting two clearly pathogenic variants, we observed high levels of IL-1β release in response to staurosporin. Importantly, these levels were never observed in the supernatants of monocytes from healthy controls even when these monocytes were exposed to doses up to 10-fold higher of PKC inhibitors (Figure S1). This result strongly suggests that PKN1/2 inhibition is sufficient to trigger Pyrin inflammasome in monocytes from FMF patients while it is not sufficient in monocytes from healthy donors. This result is in line with a recent study by Lamkanfi and colleagues who identified that a Pyrin inflammasome regulatory step relying on microtubule dynamics is lost in PBMCs from FMF patients (Van Gorp et al. 2016). The current model for the activation of the Pyrin inflammasome in healthy donors emerging from these studies, is thus a two-step activation process requiring both dephosphorylation of Pyrin and a microtubule dynamic-dependent process. As the latter step is defective in FMF patients, dephosphorylation of Pyrin following PKC superfamily inhibitor treatment (Park et al. 2016) may explain the strong response observed in monocytes from FMF patients. The current study was performed on primary cells from patients and was thus limited to the analysis of the most frequent pathogenic variants. Future studies using genetically-engineered cell lines or a larger cohort of FMF patients with diverse genotypes are required to better understand how variations in the sequence of the Pyrin protein affect PKC inhibitors responses.

The diagnosis of FMF remains clinical and based on a number of criteria that may vary depending on ethnicity and patient age (Giancane et al. 2015). Genetic testing provides a definitive confirmation in a majority of patients through the identification of two clearly pathogenic variants (Shinar et al. 2012). Yet, there is no formal diagnosis for a large proportion of patients due to the presence of variant of unknown significance, to the presence of a single (mono-allelic) variant or even to the absence of MEFV variant. Furthermore, genetic testing is routinely a matter of weeks or months. A fast FMF diagnosis may be of particular interest as indicated by the high frequency of FMF patients with a history of appendicitis or other acute abdominal surgical interventions (Lidar et al. 2008; Samli et al. 2009). In most of these surgical operations, a typical acute abdominal attack of FMF is misdiagnosed as an acute appendicitis leading to a needless and potentially harmful procedure for the patients (Berkun et al. 2007). Furthermore, FMF is associated with a lifelong daily administration of colchicine. The question of whether colchicine can be discontinued in asymptomatic FMF patients with a single or no MEFV mutations is unresolved at the moment (Sonmez et al. 2017). Although it remains to be tested on a large cohort, a functional assay assessing the hyper-responsiveness of monocytes to PKC inhibitors might help identifying patients that may stop colchicine treatment without developing novel inflammatory symptoms.

The hyper-response to PKC inhibitors was specific of monocytes from FMF patients and was not observed in patients suffering from others autoinflammatory diseases or from microbial-mediated inflammation. Importantly, the resistance to colchicine-mediated Pyrin inflammasome inhibition was also specific of FMF patients-derived monocytes (Van Gorp et al. 2016). Two different assays based on the assessment of the Pyrin inflammasome are thus available to perform a functional FMF diagnosis. The relevance and the robustness of such a functional diagnostic test remain to be tested on a large cohort of patients with different genotypes, especially to assess the potential superiority of a functional test over the current genetic test.

### REFERENCES

Throughout this application, various references describe the state of the art to which this invention pertains. The disclosures of these references are hereby incorporated by reference into the present disclosure.
Anastassiadis, Theonie, Sean W. Deacon, Karthik Devarajan, Haiching Ma, and Jeffrey R. Peterson. 2011. “Comprehensive Assay of Kinase Catalytic Activity Reveals Features of Kinase Inhibitor Selectivity.” Nature Biotechnology 29 (11): 1039-45. doi:10.1038/nbt.2017.
Ben-Chetrit, Eldad, and Isabelle Touitou. 2009. “Familial Mediterranean Fever in the World.” Arthritis Care & Research 61 (10): 1447-1453. doi:10.1002/art.24458.
Berkun, Yackov, Eli Ben-Chetrit, Aharon Klar, and Eldad Ben-Chetrit. 2007. “Peritoneal Adhesions and Intestinal Obstructions in Patients with Familial Mediterranean Fever—Are They More Frequent?” Seminars in Arthritis and Rheumatism 36 (5): 316-21. doi:10.1016/j.semarthrit.2006.11.002.
Camus, D., Y. Shinar, S. Aamar, P. Langevitz, I. Ben-Zvi, A. Livneh, and M. Lidar. 2012. ‘Silent’ Carriage of Two Familial Mediterranean Fever Gene Mutations in Large Families with Only a Single Identified Patient.” Clinical Genetics 82 (3): 288-91. doi:10.1111/j.1399-0004.2011.01785.x.
Cookson, B. T., and M. A. Brennan. 2001. “Pro-Inflammatory Programmed Cell Death.” Trends Microbiol 9 (3): 113-14.
Daniels, M., T. Shohat, A. Brenner-Ullman, and M. Shohat. 1995. “Familial Mediterranean Fever: High Gene Frequency among the Non-Ashkenazic and Ashkenazic Jewish Populations in Israel.” American Journal of Medical Genetics 55 (3): 311-14. doi:10.1002/ajmg.1320550313.
Davis, P. D., L. H. Elliott, W. Harris, C. H. Hill, S. A. Hurst, E. Keech, M. K. Kumar, G. Lawton, J. S. Nixon, and S. E. Wilkinson. 1992. “Inhibitors of Protein Kinase C. 2. Substituted Bisindolylmaleimides with Improved Potency and Selectivity.” Journal of Medicinal Chemistry 35 (6): 994-1001.
Demirkaya, Erkan, Celal Saglam, Turker Turker, Isabelle Kone-Paut, Pat Woo, Matteo Doglio, Gayane Amaryan, et al. 2016. “Performance of Different Diagnostic Criteria for Familial Mediterranean Fever in Children with Periodic Fevers: Results from a Multicenter International Registry.” The Journal of Rheumatology 43 (1): 154-60. doi:10.3899/jrheum.141249.
Dode, C., C. Pecheux, C. Cazeneuve, D. Cattan, M. Dervichian, M. Goossens, M. Delpech, S. Amselem, and G. Grateau. 2000. “Mutations in the MEFV Gene in a Large Series of Patients with a Clinical Diagnosis of Familial Mediterranean Fever.” American Journal of Medical Genetics 92 (4): 241-46.
Federici, Silvia, Giuseppina Calcagno, Martina Finetti, Romina Gallizzi, Antonella Meini, Agata Vitale, Francesco Caroli, et al. 2012. “Clinical Impact of MEFV Mutations in Children with Periodic Fever in a Prevalent Western European Caucasian Population.” Annals of the Rheumatic Diseases 71 (12): 1961-65. doi:10.1136/annrheumdis-2011-200977.
Gao, Wenqing, Jieling Yang, Wang Liu, Yupeng Wang, and Feng Shao. 2016. “Site-Specific Phosphorylation and Microtubule Dynamics Control Pyrin Inflammasome Activation.” Proceedings of the National Academy of Sciences of the United States of America 113 (33): E4857-4866. doi:10.1073/pnas.1601700113.
Giancane, Gabriella, Nienke M. Ter Haar, Nico Wulffraat, Sebastiaan J. Vastert, Karyl Barron, Veronique Hentgen, Tilmann Kallinich, et al. 2015. “Evidence-Based Recommendations for Genetic Diagnosis of Familial Mediterranean Fever.” Annals of the Rheumatic Diseases 74 (4): 635-41. doi:10.1136/annrheumdis-2014-206844.
Goldfinger, S. E. 1972. “Colchicine for Familial Mediterranean Fever.” The New England Journal of Medicine 287 (25): 1302. doi:10.1056/NEJM197212212872514.
Jeru, Isabelle, Veronique Hentgen, Emmanuelle Cochet, Philippe Duquesnoy, Gaelle Le Borgne, Emmanuel Grimprel, Katia Stankovic Stojanovic, Sonia Karabina, Gilles Grateau, and Serge Amselem. 2013. “The Risk of Familial Mediterranean Fever in MEFV Heterozygotes: A Statistical Approach.” PloS One 8 (7). doi:10.1371/journal.pone.0068431.
Jeru, Isabelle, Stephanie Papin, Sebastien L'hoste, Philippe Duquesnoy, Cecile Cazeneuve, Jacques Camonis, and Serge Amselem. 2005. “Interaction of Pyrin with 14.3.3 in an Isoform-Specific and Phosphorylation-Dependent Manner Regulates Its Translocation to the Nucleus.” Arthritis and Rheumatism 52 (6): 1848-57. doi:10.1002/art.21050.
Kogan, A., Y. Shinar, M. Lidar, A. Revivo, P. Langevitz, S. Padeh, M. Pras, and A. Livneh. 2001. “Common MEFV Mutations among Jewish Ethnic Groups in Israel: High Frequency of Carrier and Phenotype III States and Absence of a Perceptible Biological Advantage for the Carrier State.” American Journal of Medical Genetics 102 (3): 272-76.
La Regina, Micaela, Gabriella Nucera, Marialuisa Diaco, Antonio Procopio, Giovanni Gasbarrini, Cecile Notarnicola, Isabelle Kone-Paut, Isabelle Touitou, and Raffaele Manna. 2003. “Familial Mediterranean Fever Is No Longer a Rare Disease in Italy.” European Journal of Human Genetics: EJHG 11 (1): 50-56. doi:10.1038/sj.ejhg.5200916.
Lachmann, H. J., B. Sengul, T. U. Yavuzsen, D. R. Booth, S. E. Booth, A. Bybee, J. R. Gallimore, et al. 2006. “Clinical and Subclinical Inflammation in Patients with Familial Mediterranean Fever and in Heterozygous Carriers of MEFV Mutations.” Rheumatology (Oxford, England) 45 (6): 746-50. doi:10.1093/rheumatology/kei279.
Lidar, M., A. Doron, R. Kedem, A. Yosepovich, P. Langevitz, and A. Livneh. 2008. “Appendectomy in Familial Mediterranean Fever: Clinical, Genetic and Pathological Findings.” Clinical and Experimental Rheumatology 26 (4): 568-73.
Lidar, M., I. Tokov, A. Chetrit, N. Zaks, P. Langevitz, and A. Livneh. 2005. “Diagnosis Delay in Familial Mediterranean Fever (FMF): Social and Gender Gaps Disclosed.” Clinical and Experimental Rheumatology 23 (3): 357-63.
Maranon, F., K. Burns, and J. Tschopp. 2002. “The Inflammasome: A Molecular Platform Triggering Activation of Inflammatory Caspases and Processing of proIL-Beta.” Mol Cell 10 (2): 417-26.
Masters, Seth L., Vasiliki Lagou, Isabelle Jeru, Paul J. Baker, Lien Van Eyck, David A. Parry, Dylan Lawless, et al. 2016. “Familial Autoinflammation with Neutrophilic Dermatosis Reveals a Regulatory Mechanism of Pyrin Activation.” Science Translational Medicine 8 (332): 332ra45. doi:10.1126/scitranslmed.aaf1471.
Migita, Kiyoshi, Yasumori Izumi, Yuka Jiuchi, Nozomi Iwanaga, Chieko Kawahara, Kazunaga Agematsu, Akihiro Yachie, et al. 2016. “Familial Mediterranean Fever Is No Longer a Rare Disease in Japan.” Arthritis Research & Therapy 18 (July): 175. doi:10.1186/s13075-016-1071-5.
Mor, Adam, Rivka Gal, and Avi Livneh. 2003. “Abdominal and Digestive System Associations of Familial Mediterranean Fever.” The American Journal of Gastroenterology 98 (12): 2594-2604. doi:10.1111/j.1572-0241.2003.08784.x.
Omenetti, Alessia, Sonia Carta, Laura Delfino, Alberto Martini, Marco Gattorno, and Anna Rubartelli. 2014. “Increased NLRP3-Dependent Interleukin 1beta Secretion in Patients with Familial Mediterranean Fever: Correlation with MEFV Genotype.” Annals of the Rheumatic Diseases 73 (2): 462-69. doi:10.1136/annrheumdis-2012-202774.
Ozen, S., Y. Karaaslan, O. Ozdemir, U. Saatci, A. Bakkaloglu, E. Koroglu, and S. Tezcan. 1998. “Prevalence of Juvenile Chronic Arthritis and Familial Mediterranean Fever in Turkey: A Field Study.” The Journal of Rheumatology 25 (12): 2445-49.
Padeh, Shai, Avi Livneh, Elon Pras, Yael Shinar, Merav Lidar, Olga Feld, and Yackov Berkun. 2010. “Familial Mediterranean Fever in Children Presenting with Attacks of Fever Alone.” The Journal of Rheumatology 37 (4): 865-69. doi:10.3899/jrheum.090687.
Park, Yong Hwan, Geryl Wood, Daniel L. Kastner, and Jae Jin Chae. 2016. “Pyrin Inflammasome Activation and RhoA Signaling in the Autoinflammatory Diseases FMF and HIDS.” Nature Immunology, June: 914-921 doi:10.1038/ni.3457.
Samli, Hale, Fadime Mutlu Icduygu, Asuman Ozgoz, Gokhan Akbulut, Kuyas Hekimler, and Necat Imirzalioglu. 2009. “Surgery for Acute Abdomen and MEFV Mutations in Patients with FMF.” Acta Reumatologica Portuguesa 34 (3): 520-24.
Sarkisian, T., H. Ajrapetian, A. Beglarian, G. Shahsuvarian, and A. Egiazarian. 2008. “Familial Mediterranean Fever in Armenian Population.” Georgian Medical News, no. 156 (March): 105-11.
Sarrauste de Menthiere, Cyril, Stephane Terriere, Denis Pugnere, Manuel Ruiz, Jacques Demaille, and Isabelle Touitou. 2003. “INFEVERS: The Registry for FMF and Hereditary Inflammatory Disorders Mutations.” Nucleic Acids Research 31 (1): 282-85.
Shinar, Y., L. Obici, I. Aksentijevich, B. Bennetts, F. Austrup, I. Ceccherini, J. M. Costa, et al. 2012. “Guidelines for the Genetic Diagnosis of Hereditary Recurrent Fevers.” Annals of the Rheumatic Diseases 71 (10): 1599-1605. doi:10.1136/annrheumdis-2011-201271.
Sonmez, Hafize Emine, Ezgi Deniz Batu, Yelda Bilginer, and Seza Ozen. 2017. “Discontinuing Colchicine in Symptomatic Carriers for MEFV (Mediterranean FeVer) Variants.” Clinical Rheumatology 36 (2): 421-25. doi:10.1007/s10067-016-3421-8.
Sonmez, Hafize Emine, Ezgi Deniz Batu, and Seza Ozen. 2016. “Familial Mediterranean Fever: Current Perspectives.” Journal of Inflammation Research 9: 13-20. doi:10.2147/JIR.S91352.
Soriano, Alessandra, and Raffaele Manna. 2012. “Familial Mediterranean Fever: New Phenotypes.” Autoimmunity Reviews 12 (1): 31-37. doi:10.1016/j.autrev.2012.07.019.
Tamaoki, T. 1991. “Use and Specificity of Staurosporine, UCN-01, and Calphostin C as Protein Kinase Inhibitors.” Methods in Enzymology 201: 340-47.
Toplak, Natasa, Joost Frenkel, Seza Ozen, Helen J. Lachmann, Patricia Woo, Isabelle Kone-Paut, Fabrizio De Benedetti, et al. 2012. “An International Registry on Autoinflammatory Diseases: The Eurofever Experience.” Annals of the Rheumatic Diseases 71 (7): 1177-82. doi:10.1136/annrheumdis-2011-200549.
Van Gorp, Hanne, Pedro H. V. Saavedra, Nathalia M. de Vasconcelos, Nina Van Opdenbosch, Lieselotte Vande Walle, Magdalena Matusiak, Giusi Prencipe, et al. 2016. “Familial Mediterranean Fever Mutations Lift the Obligatory Requirement for Microtubules in Pyrin Inflammasome Activation.” Proceedings of the National Academy of Sciences of the United States of America, November. doi:10.1073/pnas.1613156113.
Xu, Hao, Jieling Yang, Wenqing Gao, Lin Li, Peng Li, Li Zhang, Yi-Nan Gong, et al. 2014. “Innate Immune Sensing of Bacterial Modifications of Rho GTPases by the Pyrin Inflammasome.” Nature 513 (7517): 237-41. doi:10.1038/nature13449.

1. An in vitro method for diagnosing Familial Mediterranean Fever (FMF) disease in a subject, comprising the steps of i) treating immune primary cells obtained from the subject with a NF-κB activator and then with a Protein Kinase C (PKC) inhibitor ii) detecting the level IL-1 beta secreted from the immune primary cells supernatant iii) comparing the level determined in step ii) with a reference value and iv) concluding that the subject suffers from Familial Mediterranean Fever when the level of IL1 beta determined at step ii) is higher than the reference value.

2. The in vitro method according to claim 1, wherein the immune primary cells are selected from the group consisting of peripheral blood mononuclear cells (PBMCs), white blood cells (WBCs), monocytes and neutrophils.

3. The in vitro method according to of claim 1, wherein the NF-κB activator is Lipopolysaccharides (LPS).

4. The in vitro method according to claim 1 wherein the PKC inhibitor is selected from the group consisting of Staurosporine, UCN-01 and Ro-31-8220.

5. An in vitro method for diagnosing Familial Mediterranean Fever (FMF) disease in a subject, comprising the steps of i) treating immune primary cells obtained from the subject with a Protein Kinase C (PKC) inhibitor ii) detecting the level of IL-18 secreted from the immune primary cells iii) comparing the level determined in step ii) with a reference value and iv) concluding that the subject suffers from an Familial Mediterranean Fever when level of IL-18 determined at step ii) is higher than the reference value.

6. The in vitro method according to claim 5, wherein the immune primary cells are treated with NF-κB activator prior to step i).

7. The in vitro method according to claim 6, wherein the NF-κB activator is Lipopolysaccharides (LPS).

8. The in vitro method according to claim 5, wherein the immune primary cells are selected from the group consisting of peripheral blood mononuclear cells (PBMCs), white blood cells (WBCs), monocytes and neutrophils.

9. The in vitro method according to claim 5 wherein the PKC inhibitor is selected from the group consisting of Staurosporine, UCN-01 and Ro-31-8220.

10-16. (canceled)

17. Method of treating Familial Mediterranean fever (FMF) in a subject in need thereof, comprising:
a) obtaining immune primary cells from the subject,
b) treating the immune primary cells with a NF-κB activator and then with a Protein Kinase C (PKC) superfamily inhibitor
c) detecting the level of IL1 beta secreted from the immune primary cells
d) comparing the level determined in step ii) with a reference value and
when the IL1 beta level determined at step ii) is higher than the reference value, treating the subject with a suitable treatment.

18. The method of claim 5, wherein step of detecting is performed 3 hours or less after the step of treating.

19. The method of claim 17, wherein the suitable treatment includes one or more of administering to the subject at least one of colchicine, and IL1 antagonist, a monoclonal antibody targeting interleukin-1 beta or an interleukin 1 (IL1) receptor antagonist.

20. The method of claim 17, further comprising a step of contacting the immune primary cells with an NF-κB activator prior to step b).

21. The method of claim 17, wherein the immune primary cells are selected from the group consisting of peripheral blood mononuclear cells (PBMCs), white blood cells (WBCs), monocytes and neutrophils.

22. The method of claim 20, wherein the NF-κB activator is Lipopolysaccharides (LPS).

23. The method of claim 17, wherein the PKC inhibitor is selected from the group consisting of Staurosporine, UCN-01 and Ro-31-8220.

24. The method of claim 17, wherein step of detecting is performed 3 hours or less after the step of treating.
